(19) United States
(12) Patent Application Publication
(10) Pub. No.: US 2026/0273237 A1
(43) Pub. Date: Sep. 17, 2026

(54) PFAS-FREE MEDICAL DEVICE PROTECTORS

(71) Applicant: Boston Scientific Scimed, Inc., Maple Grove, MN (US)

(72) Inventors: Joel M. WasDyke, Independence, MN (US); Joseph Thomas Delaney, JR., Minneapolis, MN (US)

(73) Assignee: Boston Scientific Scimed, Inc., Maple Grove, MN (US)

(21) Appl. No.: 19/564,753

(22) Filed: Mar. 12, 2026

Related U.S. Application Data
(60) Provisional application No. 63/770,544, filed on Mar. 12, 2025.

(51) Int. Cl. A61M 25/10

(52) U.S. Cl. CPC A61M 25/10; A61M 2025/1081

(57) ABSTRACT

The present disclosure is directed to balloon catheters including a PFAS-free balloon protector and stent delivery systems including the balloon catheters. For instance, the present disclosure is directed to a balloon catheter including a balloon and a balloon protector extending about at least a portion of the balloon, wherein the balloon protector is made from a material comprising a dispersion of a polyamide and a lubricious polyethylene, where a weight ratio of the polyamide and the lubricious polyethylene is in a range from about 8:1 to about 20:1.

### CROSS-REFERENCE TO RELATED APPLICATIONS

This application claims the benefit of priority under 35 U.S.C. § 119 of U.S. Provisional Application No. 63/770,544, filed Mar. 12, 2025, the entire disclosure of which is hereby incorporated by reference.

### TECHNICAL FIELD

The present disclosure relates to the field of medical catheters used for the delivery of medical devices such as balloons and/or stents. More specifically, the present disclosure invention relates generally to per- and polyfluoroalkyl substances (PFAS)-free medical device protectors.

### BACKGROUND

Expandable, implantable medical devices such as balloon and/or stents are utilized in a number of medical procedures and situations as are balloon and/or stent delivery assemblies. As such, their general structure and function are well known. A stent is a generally cylindrical prosthesis introduced via a catheter into a lumen of a body vessel in a configuration having a generally reduced diameter and then expanded to the diameter of the vessel. The stent may be self-expanding, for example, the stent may comprise a super elastic and/or linear elastic material such as nickel-titanium alloy (Nitinol), or it may be expandable by means of an inflatable portion of the catheter, such as a balloon. In its expanded configuration, the stent supports and reinforces the vessel walls while maintaining the vessel in an open, unobstructed condition.

Balloon expandable and balloon assisted expandable stents are expanded via outward radial pressure such as that provided by a balloon disposed underneath the stent during inflation of the balloon.

In advancing a balloon expandable stent through a body vessel to the deployment site, there are a number of important considerations, such as delivery device size, stent parameters, and/or condition of deployment site. Medical device delivery balloons may have a variety of shapes, sizes, inflation characteristics and a variety of other performance attributes. The catheter helps to atraumatically advance the system and protects the stent. The stents can be delivered into the lumen using a system which includes a catheter, with the stent supported near its distal end, and a sheath, positioned coaxially about the catheter and over the stent, to prevent abrasion between the stent and body wall as the catheter is directed through torturous body pathways.

Once the stent is located at the constricted portion of the lumen, the sheath is removed to expose the stent, which is expanded so it contacts the lumen wall. The catheter is subsequently removed from the body by pulling it in the proximal direction, through the larger lumen diameter created by the expanded prosthesis, which is left in the body.

The stent ends and/or balloons are often damaged during delivery through the catheter, or shipping of a pre-loaded delivery device. Thus, it is desirable to protect the distal and proximal ends of the stent and/or the balloons to prevent distortion of the stent, prevent damage to the balloons, prevent unintended delivery of a therapeutic drug to a site other than a target site in vivo, and/or to prevent abrasion and/or to reduce potential trauma to the vessel walls. Hence, some approaches employ stent or catheter protectors (e.g., balloon catheter protectors) to protect one or more medical device (e.g., a balloon and/or a stent). One traditional material that has been commonly used to provide a low friction surface for medical device protectors is polytetrafluoroethylene (PTFE).

### SUMMARY

In an aspect, a balloon catheter is provided. The balloon catheter comprising: a balloon; and a balloon protector extending about at least a portion of the balloon, wherein the balloon protector is made from a material comprising a dispersion of a polyamide and a lubricious polyethylene, wherein a weight ratio of the polyamide and the lubricious polyethylene is in a range from about 8:1 to about 20:1.

In some aspects, which may be used in combination with one or more aspects herein, wherein the lubricious polyethylene is a high-density polyethylene (HDPE), an ultra-high molecular weight polyethylene (UHMWPE), a maleic anhydride-grafted HDPE, a maleic anhydride-grafted LDPE, or any combination thereof.

In some aspects, which may be used in combination with one or more aspects herein, wherein the polyamide is a biocompatible polyamide, the lubricious polyethylene is a biocompatible lubricious polyethylene, or wherein the polyamide and the lubricious polyethylene are biocompatible.

In some aspects, which may be used in combination with one or more aspects herein, wherein the polyamide is selected from nylon-11, nylon-12, or a combination thereof.

In some aspects, which may be used in combination with one or more aspects herein, wherein the polyamide forms about 85 to about 95 weight percent of a total weight of the balloon protector.

In some aspects, which may be used in combination with one or more aspects herein, wherein the lubricious polyethylene is a high-density polyethylene (HDPE) or a maleic anhydride-grafted LDPE.

In some aspects, which may be used in combination with one or more aspects herein, wherein the lubricious polyethylene forms about 5 to about 15 weight percent of a total weight of the balloon protector.

In some aspects, which may be used in combination with one or more aspects herein, further comprising a compatibilizer that forms about 0.5 weight percent to about 3 weight percent of a total weight of the balloon protector.

In some aspects, which may be used in combination with one or more aspects herein, wherein the compatibilizer is a carboxylic acid-based compatibilizer.

In some aspects, which may be used in combination with one or more aspects herein, wherein the compatibilizer is a maleic anhydride-grafted Low-Density Polyethylene (LDPE), a maleic anhydride-grafted High-Density Polyethylene (HDPE), or a combination thereof.

In some aspects, which may be used in combination with one or more aspects herein, wherein a combined weight of the polyamide, the lubricious polyethylene, and the compatibilizer is equal to a total weight of the balloon protector.

In some aspects, which may be used in combination with one or more aspects herein, the lubricious polyethylene forms about 7.5 weight percent to about 9 weight percent of a total weight of the balloon protector; the polyamide forms about 90 weight percent of the total weight of the balloon protector; and the compatibilizer forms about 1 weight percent to about 2.5 weight percent of the total weight of the balloon protector.

In some aspects, which may be used in combination with one or more aspects herein, wherein the balloon protector is an elongated tubular balloon protector.

In some aspects, which may be used in combination with one or more aspects herein, wherein the balloon protector is per- and polyfluoroalkyl substances (PFAS)-free.

In some aspects, which may be used in combination with one or more aspects herein, wherein the balloon protector exhibits a comparable or reduced removal force compared to a removal force of a PFAS based balloon protector.

In another aspect, a balloon delivery catheter is provided. The balloon delivery catheter comprising: a balloon catheter including a balloon; and a balloon protector extending at least partially about the balloon, wherein the balloon protector is made from a dispersion of a polyamide, a lubricious polyethylene, and a carboxylic acid-based compatibilizer.

In some aspects, which may be used in combination with one or more aspects herein, wherein: the polyamide is Grilamid; the lubricious polyethylene is a HDPE and the carboxylic acid-based compatibilizer is a maleic anhydride-grafted low-density polyethylene (LDPE).

In some aspects, which may be used in combination with one or more aspects herein, wherein the balloon protector is per- and polyfluoroalkyl substances (PFAS)-free, and the balloon protector exhibits a reduced removal force compared to a removal force of a PFAS based balloon protector.

In another aspect, a balloon delivery system is provided. The balloon delivery system comprising: a balloon catheter including a balloon and outer sheath; and an elongated tubular balloon protector extending at least partially about the balloon; and wherein the elongated tubular balloon protector is made from a dispersion of a polyamide, a high-density polyethylene (HDPE), and a carboxylic acid based compatibilizer, wherein a combined weight of the polyamide, the HDPE, and the carboxylic acid based compatibilizer are equal to a total weight of the elongated tubular balloon protector, wherein the elongated tubular balloon protector is per- and polyfluoroalkyl substance (PFAS)-free, wherein the elongated tubular balloon protector has a rough, non-uniform exterior surface as compared to an exterior surface of a PFAS based balloon protector, and wherein the elongated tubular balloon protector exhibits a reduced removal force compared to a removal force of the PFAS based balloon protector.

In some aspects, which may be used in combination with one or more aspects herein, wherein: the carboxylic acid based compatibilizer is a maleic anhydride-grafted low-density polyethylene (LDPE), and the maleic anhydride-grafted LDPE forms about 1 weight percent to about 2.5 weight percent of a total weight of the elongated tubular balloon protector; the polyamide forms about 7.5 weight percent to about 9 weight percent of the total weight of the elongated tubular balloon protector; and the HDPE forms about 90 weight percent of the total weight of the elongated tubular balloon protector.

The above summary of some example embodiments is not intended to describe each disclosed embodiment or every implementation of the invention.

### BRIEF DESCRIPTION OF THE DRAWINGS

FIG. 1 is a perspective side view showing an example of a balloon catheter including a per- and polyfluoroalkyl substances (PFAS)-free medical device protector (balloon protector) of the present disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification. All numeric values are herein assumed to be modified by the term “about”, whether or not explicitly indicated. The term “about” generally refers to a range of numbers that one of skill in the art would consider equivalent to the recited value (i.e., having the same function or result). For instance, the term “about” may include values that are within 1 percent, 2 percent, 5 percent, 10 percent of a recited value. In many instances, the term “about” may be indicative as including numbers that are rounded to the nearest significant FIGURE.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

It is generally known to provide substrates, for instance medical devices or parts of such devices, with a coating for the purpose of reducing the friction between medical devices themselves or between a medical device and a tissue when the device is introduced in an aqueous environment, such as the human body, or within another medical device. Such coatings have also been referred to as lubricious or “slippery” coatings. Catheters and other medical devices used for introduction in blood vessels, body conduits and the like and guide wires used with such devices are examples of articles which may be provided with coatings. Catheters for balloon angioplasty and biopsy are specific examples of such catheters. Other illustrative medical devices may include, but are not limited to, stents, embolic filters, implantable devices, treatment devices, diagnostic devices, guide catheters, sheaths, etc.

A stent is an implantable medical device introduced into a body lumen and is well known in the art. Typically, a stent is implanted in a blood vessel at the site of a stenosis or aneurysm endoluminally, i.e., by so-called “minimally invasive techniques” in which the stent, in a radially reduced configuration, optionally restrained in a radially compressed configuration by a sheath and/or catheter, is delivered by a stent delivery system or “introducer” to the site where it is required. The introducer can enter the body from an access location outside the body, such as through the patient's skin, or by a “cut down” technique in which the entry vessel is exposed by minor surgical means.

Stents are often inserted during percutaneous transluminal coronary angioplasty or PTCA procedures to reduce the risk of restenosis.

A further means of reducing the risk of restenosis is to treat the stent with a therapeutic agent that assists in preventing restenosis. One method is to bioengineer coatings that release the therapeutic agent in a controlled manner, either from the coating itself, or from the stent. Of course, therapeutic agents may be added for other reasons as well such as to decrease inflammation or to have an antibiotic effect, for example. Some of these coatings are susceptible to environmental exposure such as environmental moisture, oxygen and light, for example. For example, coatings, also referred to in the art as excipients, that may include any of a variety of bioerodable or biodegradable polymers such as those comprising l-lactide or d,l-lactide, with or without glycolide, can be hydrolytically unstable when subjected to atmospheric moisture. Some therapeutic agents are also hydrolytically unstable, for example, Everolimus. A variety of packaging methods have been developed for just these reasons.

Expandable, implantable medical devices such as balloon and/or stents are utilized in a number of medical procedures and situations as are balloon and/or stent delivery assemblies. As such, their general structure and function are well known.

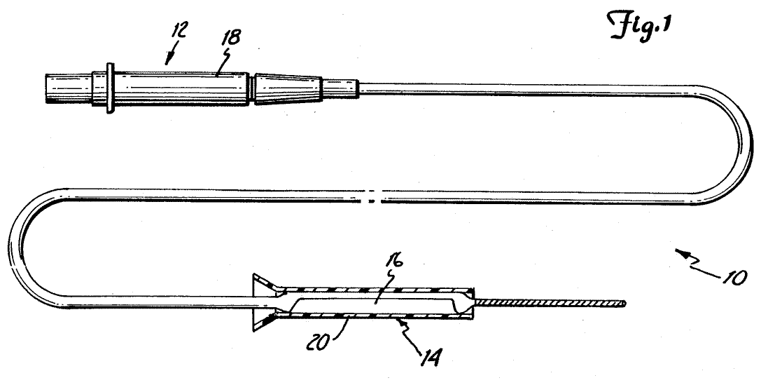

For instance, a balloon catheter 10 is shown in FIG. 1. The balloon catheter 10 has a proximal end 12 and a distal end 14. At the distal end 14 is an inflatable balloon 16. At the proximal end 12 is an inflation manifold 18. The inflatable balloon 16 is encased by a balloon protector 20. The balloon protector 20 is normally applied to distal end 14 of catheter 10 during the manufacturing process, and remains in place during shipping and storage to prevent damage to the balloon 16. While FIG. 1 illustrates the balloon protector 20 as having a particular shape and size and being in a particular relationship with other components in the catheter 10, the shape, size, and/or relative configuration or location of the balloon protector 20 and/or the other components in FIG. 1 can be varied. For instance, the balloon protector can be configured as a removable balloon protector e.g., having a longitudinally extending slit configured to permit the balloon protector 20 to be removed from the catheter 10, among other possibilities.

One traditional material that has been commonly used to provide a low friction surface, for instance in stent or balloon protectors, is polytetrafluoroethylene (PTFE). However, because of its very low coefficient of friction, it is difficult to wet out the surface of PTFE. Consequently, it can be difficult to adhere other polymers to PTFE, making it difficult to use as a low friction coating. Furthermore, PTFE is very difficult to process. Additionally, use of PTFE along with other per- and polyfluoroalkyl substances (PFAS) may increasingly be subject to various regulatory considerations, environmental concerns, and/or may otherwise be costly. Hence, the use of PFAS (e.g., PTFE) based stent or balloon protectors may be undesirable.

As such, the present disclosure pertains to systems and medical devices with PFAS-free medical device protectors (e.g., balloon and/or stent protectors) that exhibit comparable or even improved performance (e.g., at least in terms of removal force) as compared to PFAS based medical device protectors (e.g., PTFE containing medical device protectors). The comparable or improved performance of the PFAS-free medical device protectors herein can yield various benefits such as easing the deliverability, placement, and/or removal of one or more medical device, and yet the medical device protectors described herein are PFAS-free.

Moreover, the PFAS-free medical device protectors described herein desirably are colorable (as compared to some other medical device protectors that are manufactured with an inherent color attributable to the materials forming the medical device protectors), are translucent (e.g., to promote visibility, facilitate light based-curing, and/or facilitate light-based medical treatments or procedure), and are dimensionally stable and thus are not prone to an appreciable reduction in lubricity performance (e.g., that would otherwise correspond to an appreciable increase in removal force associated of the medical device protectors over time). For example, medical device protectors formed of the translucent materials described herein can facilitate visual inspection of a material or device (e.g., a balloon such as drug-coated balloon) disposed within the translucent medical device protectors e.g., to visually assess the structural integrity of the balloon and/or a material (e.g., a drug-eluting coating) disposed on the balloon. For instance, the dispersions of material (e.g., a dispersion comprising a polyamide and lubricious polyethylene such as HDPE, UHMWPE and/or a maleic anhydride-grafted LDPE) that form the medical device protectors described herein are phase stable and are thus not prone to degradation or an appreciable reduction in lubricity associated with longer indwell times (e.g., as compared to an increase in removal force typically associated with longer indwell times of PTFE-based medical device protectors).

In some embodiments, various balloon expandable prostheses may be employed in conjunction with the balloon catheters of the present disclosure. A stent, stent covered graft, or other stent/graft combinations may be employed as are known in the art. Among the various stents that may be employed, there are a host of geometries and materials of construction to choose from, as is known in the art. Some suitable stent metallic materials include, but are not necessarily limited to, stainless steel, tantalum, tungsten, nickel-titanium alloys such as those possessing shape memory properties commonly referred to as nitinol, nickel-chromium alloys, nickel-chromium-iron alloys, cobalt-chromium-nickel alloys, or other suitable metals, or combinations or alloys thereof. Some suitable stent polymeric materials include, but are not necessarily limited to polyamide, polyether block amide, polyethylene, polypropylene, polyvinylchloride, polyurethane, polysulfone, and copolymers, blends, mixtures or combinations thereof.

The balloon 16 may be composed of any thermoplastic polymer, or polymers, suitable for use as a medical balloon. The balloon 16 may be a unitary body of an individual material or may be a combination of various materials with various characteristics such as those to provide additional elasticity in a middle arching section. Such thermoplastic polymers include, but are not limited to: polyethylene teraphtholate (PET), polybutylene teraphtholate (PBT), PEBAX™, Nylon™, polyurethane, polyester-polyether block copolymer such as ARNITEL™, polyolefin and polyolefin compounds. In addition, the balloon 16 may be manufactured by any means appropriate for manufacturing a medical balloon as known in the art. The balloon 16 may have a variety of geometries as known in the art. The balloon 16 may be made from typical angioplasty balloon materials including polymers such as polyethylene terephthalate (PET), polyetherimide (PET), polyethylene (PE), etc. Some other examples of suitable polymers, including lubricious polymers, may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, and a polyether-ester elastomer such as ARNITEL® available from DSM Engineering Plastics).

Additional examples of suitable polymers include polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, nylons such as polyether block amide (PEBA, for example, available under the trade name PEBAX®), silicones, Marlex high-density polyethylene, Marlex low-density polyethylene, linear low density polyethylene (for example, REXELL®), polyetheretherketone (PEEK), polyimide (PI), polyphenylene sulfide (PPS), polyphenylene oxide (PPO), polysulfone, nylon, perfluoro (propyl vinyl ether) (PFA), other suitable materials, or mixtures, combinations, copolymers thereof, polymer/metal composites, and the like.

The balloon 16 can be expandable. For instance, the balloon 16 can be configured initially in an unexpanded or delivery configuration and can be configured to expand in vivo to an expanded or deployed configuration in vivo.

As illustrated in FIG. 1, the balloon protector 20 can be manifested as an elongated tubular member, such as an elongated tubular member having a flared or wider proximal end region (e.g., as illustrated in FIG. 1). The elongated tubular member can have a lumen formed therein that extends from a proximal end to a distal end of the balloon protector 20. Alternatively, the balloon protector 20 may be a clamp, washer, necking die, ring, clamshell or the like.

The shape and/or a size (e.g., a diameter) of the balloon protector 20 can vary, for instance, depending on the size of the balloon 16 and the balloon catheter 10. In some embodiments, the balloon protector 20 can be formed of a unitary member such as being an individual elongated tubular member. In some embodiments, the balloon protector 20 can have a uniform diameter (e.g., a uniform outer and/or inner diameter) along a longitudinal length of the balloon protector. However, in some embodiments, the diameter of the balloon protector 20 can vary along a longitudinal length of the balloon protector 20.

The balloon protector 20 may be made from a variety of materials, as detailed herein, which will not damage, deteriorate, react or contaminate the balloon 16, the stent or the balloon catheter 10. The balloon protector 20 includes a lubricious and smooth inner surface to allow the protector to move over the balloon 16 without damaging it and/or provide a low removal force of the balloon protector 20. The balloon catheter can be disposed within a lumen of another device such as an introducer sheath.

In some embodiments, a material forming the balloon protector 20 may comprise a dispersion of a polyamide and a lubricious polyethylene material such as a high-density polyethylene (HDPE) or an ultra-high density polyethylene (UHMWPE). As used herein, a dispersion refers to a multi-material system including at least two materials where one material (e.g., a dispersed phase formed of the lubricious polyethylene materials herein such as HDPE or UHMWPE) is distributed within another material (e.g., a continuous phase formed of the polyamides herein). The dispersed material is typically present as fine particles (e.g., polymeric crystals or polymeric spherulites) and the continuous phase is the medium in which these particles are spread. For example, the dispersed material (e.g., a lubricious polyethylene such as HDPE or UHMWPE) can be manifested as disordered or ordered regions of spherulites which are present in the amounts described herein in the continuous phase (e.g., a polyamide).

In some embodiments, the balloon protector 20 formed from the polyamide and the lubricious polyethylene (e.g., HDPE or UHMWPE) dispersion can have a non-uniform or roughened surface. For instance, the surface of the balloon protector 20 can have various protrusions (e.g., spherulites of HDPE or UHMWPE) that project a distance from (radially outward from) the outer surface of the continuous phase. This non-uniform surface can have a degree of surface roughness that is greater than the surface roughness associated with traditional (e.g., PFAS-based) balloon protectors. Without wishing to be bound by theory, it is believed the non-uniform or roughened outer surface of the balloon protector 20 may desirably promote aspects herein such as reducing an amount of removal force of the balloon protector 20. For instance, only or substantially only the spherulites of HDPE or UHMWPE on the outer surface of the balloon protector 20 may contact an adjacent surface (e.g., of the balloon, stent, and/or the catheter) and thereby yield a reduction in frictional forces between the adjacent surface(s) and the outer surface of the balloon protector 20.

In some embodiments, the polyamide is biocompatible, the lubricious material such as HDPE or UHMWPE is biocompatible, or wherein the polyamide and the lubricious material are biocompatible. As used herein biocompatibility refers to meeting applicable biocompatibility standards for medical devices such as those in ISO 10993-1:2009.

Examples of polyamide materials useful in the present disclosure include nylon-11, nylon-12 (e.g., such as GRILAMID®; available from ENTEC POLYMERS and/or GRIVORY), or a combination thereof. Stated differently, in some embodiments, the polyamide is selected from nylon-11, nylon-12, or a combination thereof. For instance, in some embodiments, the polyamide can comprise GRILAMID®. In some embodiments, the polyamide can consist essentially of GRILAMID®. In such instances, the polyamide can include one or more different types of polyamides than GRILAMID®. In some embodiments, the polyamide can consist of GRILAMID™. As used herein, GRILAMID® can refer to any or more of GRILAMID® L; available from ENTEC POLYMERS, GRILAMID® 1S; available from ENTEC POLYMERS, GRILAMID® 2S; available from ENTEC POLYMERS, GRILAMID® 2D; available from ENTEC POLYMERS, GRILAMID® TR; available from ENTEC POLYMERS, and L25 GRILAMID®; available from GRIVORY. For instance, in some embodiments the polyamide is L25 GRILAMID®. Stated differently, in some embodiments the polyamide is an individual polyamide manifested as L25 GRILAMID®. L25 GRILAMID® is a polyamide 12 without additives or stabilizers. L25 GRILAMID® is biocompatible and offers the lowest moisture absorption of any commercially available nylon, thus providing excellent dimensional stability with mechanical properties virtually unaffected by environmental humidity.

In some embodiments, the polyamide can form about 85 to about 95 weight percent of a total weight of the balloon protector 20. That is, the polyamide can form about 85 to about 95 weight percent of a total weight of the balloon protector 20. All individual values and sub-ranges from about 85 to about 95 weight percent are included. For instance, the polyamide can form about 85, about 86, about 87, about 88, about 89, about 90, about 91, about 92, about 93, about 94, or about 95 weight percent of a total weight of the balloon protector 20. For instance, the polyamide can form about 90 weight percent of a total weight of the balloon protector 20.

Examples of lubricious polyethylene materials useful in the present disclosure include high density polyethylene (HDPE), ultra-high molecular weight polyethylene (UHMWPE), maleic anhydride-grafted HDPE, maleic anhydride-grafted LDPE, and any combination thereof. In some embodiments, the lubricious polyethylene can be a high density polyethylene (HDPE), a maleic anhydride-grafted HDPE, a maleic anhydride-grafted LDPE, and any combination thereof. In some embodiments, the lubricious polyethylene can be a high density polyethylene (HDPE), a maleic anhydride-grafted LDPE, or a combination thereof.

In some embodiments, the lubricious polyethylene can be a HDPE such as the examples of suitable HDPE described herein. In some embodiments, the lubricious polyethylene can be a maleic anhydride-grafted HDPE. In some embodiments, the lubricious polyethylene polymer can be a UHMWPE. In some embodiments, the lubricious polyethylene polymer can be a maleic anhydride-grafted LDPE. As used herein, the “maleic anhydride-grafted LDPE” refers a “maleic anhydride-grafted LDPE compatibilizer” such as those detailed herein.

An example of a suitable UHMWPE is LY1040®, available from MITSUI CHEMICALS. Examples of suitable HDPE include LM6007® available from LYONDELLBASELL, INEOS® A60-70-162, available from INEOS, and BORMED® HE2581-PH, available from BOREALIS. The lubricious polyethylene polymer can have a weight average molecular weight from about 100,000 Daltons to about 11,000,000 Daltons. All individual values and sub-ranges from about 100,000 Daltons to about 11,000,000 Daltons are included. For instance, the lubricious polyethylene polymer can have a weight average molecular weight in a range having a lower value of about 100,000 Daltons, about 200,000 Daltons, about 250,000 Daltons, about 500,000 Daltons, about 1,000,000 Daltons, about 2,000,000 Daltons, or about 3,000,000 Daltons and an upper value of about 11,000,000 Daltons, about 10,000,000 Daltons, about 9,000,000 Daltons, about 8,000,000 Daltons, about 7,000,000 Daltons, about 6,000,000 Daltons, about 5,000,000 Daltons, or about 4,000,000 Daltons, among other possibilities. In some embodiments, the lubricious polyethylene polymer can be a HDPE having a weight average molecular weight in a range from about 100,000 to about 250,000 Daltons. In some embodiments, the lubricious polyethylene polymer can be a UHMWPE having a weight average molecular weight that is greater than about 3,000,000 Daltons, 4,000,000 Daltons, greater than about 5,000,000 Daltons, greater than about 6,000,000 Daltons, greater than about 7,000,000 Daltons, greater than about 8,000,000 Daltons, greater than about 9,000,000 Daltons, or greater than about 10,000,000 Daltons. In some embodiments, the lubricious polyethylene polymer can have a weight average molecular weight of about 4,000,000 Daltons. In some embodiments, the lubricious polyethylene polymer can include a UHMWPE with a molecular weight range in a range from about 4 million Daltons to about 6 million Daltons.

The lubricious polyethylene can form about 5 to about 15 weight percent of a total weight of the balloon protector 20. Stated differently, the lubricious polyethylene can be about 5 to about 15 weight percent of a total weight of dispersion of material forming the balloon protector 20. All individual values and subranges from about 5 to about 15 weight percent are included. For instance, the lubricious polyethylene can from about 5, about 6, about 7, about 8, about 9, about 10, about 11, about 12, about 13, about 14, or about 15 weight percent a total weight of the balloon protector 20. In some embodiments, the lubricious polyethylene can be present in an amount in range from about 10 to about 7.5 weight percent or about 10 to about 1 weight percent of a total weight of the balloon protector 20.

A weight ratio of the polyamide and the HDPE or the UHMWPE in the dispersion of material forming the balloon protector 20 is in a range from about 8:1 to about 20:1. All individual values and sub-ranges from about 8:1 to about 20:1 are included. For instance, the weight ratio of the polyamide to HDPE or the UHMWPE in the dispersion of material forming the balloon protector 20 can be about 8:1, about 9:1, about 10:1, about 11:1, about 12:1, about 13:1, about 14:1, about 15:1, about 16:1, about 17:1, about 18:1, about 19:1, or about 20:1. For example, the weight ratio of the polyamide to HDPE or the UHMWPE in the dispersion of material forming the balloon protector 20 is about 9:1, about, 10:1, about 10:1, or 11:1. In some embodiments, the weight ratio of the polyamide to HDPE or the UHMWPE in the dispersion of material forming the balloon protector 20 is about 12:1. In some embodiments, the weight ratio of the polyamide to HDPE or the UHMWPE in the dispersion of material forming the balloon protector 20 is about 11:1. In some embodiments, the weight ratio of the polyamide to HDPE or the UHMWPE in the dispersion of material forming the balloon protector 20 is about 10:1. In some embodiments, the weight ratio of the polyamide to the lubricious polyethylene such as HDPE or UHMWPE in the dispersion of material forming the balloon protector 20 is about 9:1.

In some embodiments, a polyamide and the lubricious polyethylene (e.g., HDPE or a maleic anhydride-grafted LDPE) dispersion can form an entire weight of balloon protector 20. Stated differently, the balloon protector 20 can be formed of only polyamide and the lubricious polyethylene in the dispersion of material.

However, in some embodiments, the balloon protectors herein can include additional components (e.g., a compatibilizer or additives), as described herein. In such embodiments, a combined weight of the polyamide and the lubricious polyethylene can be at least 95 weight percent, at least 96 weight percent, at least 97 weight percent, at least 98 weight percent, or at least 99 weight percent of a total weight of the balloon protector 20. In such instances a balance of the weight of the balloon protector 20 can be formed by a compatibilizer and/or one or more additives.

However, the polyamide and lubricious polyethylene dispersion can additionally include a compatibilizer. For instance, the materials forming the balloon protector 20 can include the polyamide, the lubricious polyethylene, and a compatibilizer. The compatibilizer can be a carboxylic acid-based compatibilizer. As used herein, a carboxylic acid-based compatibilizer refers to a compound including one or more carboxylic acid groups. For instance, the carboxylic acid-based compatibilizer can be a maleic anhydride-grafted LDPE such as ReZilok® R 101 and/or a maleic anhydride-grafted HDPE. The presence of the carboxylic acid groups in the compatibilizer may provide sites for chemical bonding, crosslinking, or improved adhesion between the polyamide and the lubricious polyethylene components in the polyamide and lubricious polyethylene dispersions herein. For instance, the compatibilizer may mitigate any potential phase separation between the polyamide particles and the lubricious polyethylene particles in the polyamide and lubricious polyethylene dispersions herein. The resulting enhancement in chemical bonding, crosslinking, and/or improved adhesion can promote aspects such as reducing the removal forces associated with the medical device protectors (e.g., balloon protectors).

The compatibilizer can form about 0.5 weight percent to about 3.0 weight percent of a total weight of the balloon protector 20. All individual values and sub-ranges from about 0.5 weight percent to about 3.0 weight percent are included. For instance, the compatibilizer can form about 0.5, about 1.0, about 1.5, about 2.0, about 2.5, or about 3.0 weight percent of a total weight of the balloon protector 20. In some embodiments, compatibilizer can form about 1.0 weight percent to about 2.5 weight percent of a total weight of the balloon protector 20. In some embodiments, the compatibilizer can form about 2.5 weight percent of a total weight of the of the balloon protector 20. In some embodiments, the compatibilizer can form about 1.0 weight percent of a total weight of the balloon protector 20.

A combined weight of the polyamide, the lubricious polyethylene, and the compatibilizer can be equal to a total weight of the balloon protector 20. However, in some embodiments the balloon protectors herein can include additional components (e.g., additives), as described herein. In such embodiments, a combined weight of the polyamide, the lubricious polyethylene, and the compatibilizer can be equal to at least 95 weight percent, at least 96 weight percent, at least 97 weight percent, at least 98 weight percent, or at least 99 weight percent of a total weight of the balloon protector 20. In such instances a balance of the weight of the balloon protector 20 can be formed by one or more additives.

In some embodiments, the lubricious polyethylene can form about 7.5 weight percent to about 9 weight percent of a total weight of the balloon protector, the polyamide can form about 90 weight percent of the total weight of the balloon protector; and the compatibilizer can form about 2.5 weight percent to about 1 weight percent of the total weight of the balloon protector. For instance, the lubricious polyethylene can form about 7.5 weight percent of a total weight of the balloon protector; the polyamide can form about 90 weight percent of the total weight of the balloon protector, and the compatibilizer forms about 2.5 weight percent of the total weight of the balloon protector. In some embodiments, the lubricious polyethylene can form about 9.0 weight percent of a total weight of the balloon protector; the polyamide can form about 90 weight percent of the total weight of the balloon protector, and the compatibilizer forms about 1.0 weight percent of the total weight of the balloon protector.

In some embodiments, the carboxylic acid based compatibilizer is a maleic anhydride-grafted LDPE. In such embodiments, the polyamide can form about 7.5 weight percent to about 9 weight percent of a total weight of the balloon protector, the HDPE or UHMWPE can form about 90 weight percent of the total weight of the balloon protector, and the maleic anhydride-grafted LDPE can form about 2.5 weight percent to about 1 weight percent of the total weight of the balloon protector. In some embodiments, the polyamide is GRILAMID®, the lubricious polyethylene is UHMWPE, and the carboxylic acid-based compatibilizer is a maleic anhydride-grafted LDPE.

The balloon protectors described herein are PFAS-free. For instance, unlike some previous balloon protectors, the balloon protectors herein are fluorinated ethylene propylene (FEP)-free and PTFE-free. As used herein, being PFAS-free generally refers to including no appreciable or detectable amount of PFAS. For instance, the balloon protectors herein can include 0 weight percent PFAS based on a total weight of the balloon protectors.

Optional additives that may be used in the balloon protectors herein include colorants, flow or viscosity modifiers, antioxidants, coupling agents, surfactants, and/or therapeutic agents. Any such additives may be incorporated into the balloon protectors at amounts of 10 weight percent or less (e.g., ranging from 10 weight percent to 5 weight percent to 2 weight percent to 1 weight percent to 0.5 weight percent or less), based on the dry weight (e.g., excluding solvent, if present) of the balloon protectors described herein.

Aspects of the disclosure are illustrated by the following non-limiting examples.

### Examples 1-5 (EX 1-5) and Comparative Example 1 (CE 1)

Unless otherwise indicated, the materials herein were procured from SIGMA-ALDRICH.

The types and amounts of materials used to form the balloon protectors of each of examples 1-5 and the comparative example 1 are indicated in Table 1.

TABLE 1

| Balloon | | | | | | |
|---|---|---|---|---|---|---|
| protector | | | | | | |
| material(s) | | | | | | |
| (weight | | | | | | |
| percentage) | CE 1 | EX 1 | EX 2 | EX 3 | EX 4 | EX 5 |
| PTFE | 100 | — | — | — | | — |
| Polyamine | — | 90 | 90 | 90 | 90 | 90 |
| HDPE | — | — | 10 | 7.5 | 7.5 | 9 |
| Maleic | — | 10 | — | 2.5 | 2.5 | 1.0 |
| anhydride- | | | | | | |
| grafted LDPE | | | | | | |
| compatibilizer | | | | | | |

Examples 1-5 and the comparative example 1 were formed as follows.

Comparative Example 1 (CE 1)-PFAS-based balloon protector): A PTFE coated balloon protector (internal diameter of 0.074 inches; available from ZEUS) was provided as comparative example 1 (CE 1).
Example 1 (EX 1)—A PFAS-free balloon protector formed of polyamide (90 weight percent) and a maleic anhydride-grafted LDPE compatibilizer (10 weight percent) was made by extruding the combination (e.g., mixture) of the polyamide and the maleic anhydride-grafted LDPE compatibilizer via traditional extrusion techniques.
Example 2 (EX 2)—A PFAS-free balloon protector was formed of polyamide (90 weight percent) and HDPE (10 weight percent) using the same formation method as EX 1.
Example 3 (EX 3)—A PFAS-free balloon protector was formed of polyamide (90 weight percent), HDPE (7.5 weight percent), and a maleic anhydride-grafted LDPE compatibilizer (2.5 weight percent) using the same formation method as EX 1.
Example 4 (EX 4)—A PFAS-free balloon protector was formed of polyamide, HDPE (7.5 weight percent), and a maleic anhydride-grafted LDPE compatibilizer (2.5 weight percent) using the same formation method as EX 1.
Example 5 (EX 5)—A PFAS-free balloon protector was formed of polyamide (90 weight percent), HDPE (9.0 weight percent), and a maleic anhydride-grafted LDPE compatibilizer (1.0 weight percent) using the same formation method as EX 1.

CE 1 and EX 1-5 were each tested for removal force. To test for removal force, the balloon protectors of CE 1 and EX 1-5 are secured by a clamping mechanism. The force required to pull the balloon protectors of CE 1 and EX 1-5 through an orifice in the clamp were measured. Hence, removal force of the balloon protectors of CE 1 and EX 1-5 was determined based on observed frictional forces of the materials undertest for each of a quantity (e.g., three) tests. An average (mean) of each of the quantity of tests was also determined. Lower frictional force test results indicate improved lubricity and also an improved (reduced) removal force.

Table 2 indicates an average amount of force required (pounds) to pull the respective balloon protectors of CE 1 and EX 1-5 through the orifice in the clamp.

TABLE 2

| Removal Force | CE 1 | EX 1 | EX 2 | EX 3 | EX 4 | EX 5 |
|---|---|---|---|---|---|---|
| Average removal force of | .924 | .181 | .159 | .193 | .327 | .176 |
| Tests (pounds) | | | | | | |

As indicated in Table 2, each of the EX 1-5 exhibited an improved (reduced) average removal force as compared to the average removal force of CE 1. For instance, each of EX 1-5 exhibits at least a 60 percent reduction in average removal force as compared to the average removal force of CE 1. This indicates that the PFAS-free balloon protectors of EX 1-5 are viable alternatives to, or even improvements upon, PFAS-containing balloon protectors such as the PFAS containing balloon protector of CE 1. For instance, the PFAS-free balloon protectors of EX 1-5 that exhibit reduced removal force may contribute to improved overall usability of the balloon protectors such as promoting ease of delivery to, placement in, and/or removal of the balloon protectors from a body lumen. Without wishing to be bound the theory, the desirable removal force performance observed by the PFAS-free balloon protectors in EX 1-5 is believed to be at least partially attributable to the synergistic combination of polyamide and lubricious polyethylene (e.g., HDPE, maleic anhydride-grafted HDPE, or maleic anhydride-grafted LDPE) in the relative proportions described herein (e.g., ranging from approximately 20:1 to 8:1) and/or the resulting surface morphology of the PFAS-free balloon protectors formed therefrom.

For instance, each of the PFAS-free balloon protectors in Examples 1-5 exhibited a rough, non-uniform outer surface (e.g., as observed visually with the aid of a microscope). As mentioned, this non-uniform surface can have a degree of surface roughness than is greater than the surface roughness associated with traditional (e.g., PFAS-based) balloon protectors. Without wishing to be bound by theory, it is believed the non-uniform or roughened outer surface of the balloon protector 20 may desirably promote aspects herein such as reducing an amount of removal force of the balloon protectors of EX 1-5. For instance, only or substantially only the spherulites of HDPE or UHMWPE on the outer surface of the balloon protector 20 may contact an adjacent surface (e.g., of the balloon, stent, and/or the catheter) and thereby yield a reduction in frictional forces between the adjacent surface(s) and the outer surface of the balloon protectors of EX 1-5.

Having described the preferred embodiments herein, it should now be appreciated that variations may be made thereto without departing from the contemplated scope of the disclosure. Accordingly, the preferred embodiments described herein are deemed illustrative rather than limiting, the true scope of the disclosure being set forth in the claims appended hereto.

What is claimed is:

1. A balloon catheter comprising:
a balloon; and
a balloon protector extending about at least a portion of the balloon, wherein the balloon protector is made from a material comprising a dispersion of a polyamide and a lubricious polyethylene, wherein a weight ratio of the polyamide and the lubricious polyethylene is in a range from about 8:1 to about 20:1.

2. The balloon catheter of claim 1, wherein the lubricious polyethylene is a high-density polyethylene (HDPE), an ultra-high molecular weight polyethylene (UHMWPE), or a maleic anhydride-grafted LDPE.

3. The balloon catheter of claim 1, wherein the polyamide is a biocompatible polyamide, the lubricious polyethylene is a biocompatible lubricious polyethylene, or wherein the polyamide and the lubricious polyethylene are biocompatible.

4. The balloon catheter of claim 1, wherein the polyamide is nylon-11, nylon-12, or a combination thereof.

5. The balloon catheter of claim 1, wherein the polyamide forms about 85 to about 95 weight percent of a total weight of the balloon protector.

6. The balloon catheter of claim 1, wherein the lubricious polyethylene is a high-density polyethylene (HDPE) or a maleic anhydride-grafted LDPE.

7. The balloon catheter of claim 1, wherein the lubricious polyethylene forms about 5 to about 15 weight percent of a total weight of the balloon protector.

8. The balloon catheter of claim 1, further comprising a compatibilizer that forms about 0.5 weight percent to about 3 weight percent of a total weight of the balloon protector.

9. The balloon catheter of claim 8, wherein the compatibilizer is a carboxylic acid-based compatibilizer.

10. The balloon catheter of claim 9, wherein the compatibilizer is a maleic anhydride-grafted Low-Density Polyethylene (LDPE), a maleic anhydride-grafted High-Density Polyethylene (HDPE), or a combination thereof.

11. The balloon catheter of claim 9, wherein a combined weight of the polyamide, the lubricious polyethylene, and the compatibilizer is equal to a total weight of the balloon protector.

12. The balloon catheter of claim 9, wherein:
the lubricious polyethylene forms about 7.5 weight percent to about 9 weight percent of a total weight of the balloon protector;
the polyamide forms about 90 weight percent of the total weight of the balloon protector; and
the compatibilizer forms about 1 weight percent to about 2.5 weight percent of the total weight of the balloon protector.

13. The balloon catheter of claim 1, wherein the balloon protector is an elongated tubular balloon protector.

14. The balloon catheter of claim 1, wherein the balloon protector is per- and polyfluoroalkyl substances (PFAS)-free.

15. The balloon catheter of claim 14, wherein the balloon protector exhibits a comparable or reduced removal force compared to a removal force of a PFAS based balloon protector.

16. A balloon delivery catheter comprising:
a balloon catheter including a balloon; and
a balloon protector extending at least partially about the balloon, wherein the balloon protector is made from a dispersion of a polyamide, a lubricious polyethylene, and a carboxylic acid-based compatibilizer.

17. The balloon catheter of claim 16, wherein:
the polyamide is Grilamid;
the lubricious polyethylene is a HDPE and
the carboxylic acid-based compatibilizer is a maleic anhydride-grafted low-density polyethylene (LDPE).

18. The balloon delivery catheter of claim 16, wherein the balloon protector is per- and polyfluoroalkyl substances (PFAS)-free, and the balloon protector exhibits a reduced removal force compared to a removal force of a PFAS based balloon protector.

19. A balloon delivery system comprising:
a balloon catheter including a balloon and outer sheath; and
an elongated tubular balloon protector extending at least partially about the balloon; and wherein the elongated tubular balloon protector is made from a dispersion of a polyamide, a high-density polyethylene (HDPE), and a carboxylic acid based compatibilizer, wherein a combined weight of the polyamide, the HDPE, and the carboxylic acid based compatibilizer are equal to a total weight of the elongated tubular balloon protector, wherein the elongated tubular balloon protector is per- and polyfluoroalkyl substance (PFAS)-free, wherein the elongated tubular balloon protector has a rough, non-uniform exterior surface as compared to an exterior surface of a PFAS based balloon protector, and wherein the elongated tubular balloon protector exhibits a reduced removal force compared to a removal force of the PFAS based balloon protector.

20. The balloon delivery system of claim 19, wherein:
the carboxylic acid based compatibilizer is a maleic anhydride-grafted low-density polyethylene (LDPE), and the maleic anhydride-grafted LDPE forms about 1 weight percent to about 2.5 weight percent of a total weight of the elongated tubular balloon protector;
the polyamide forms about 7.5 weight percent to about 9 weight percent of the total weight of the elongated tubular balloon protector; and
the HDPE forms about 90 weight percent of the total weight of the elongated tubular balloon protector.
